(19) United States
(12) Patent Application Publication
(10) Pub. No.: US 2013/0084584 A1
(43) Pub. Date: Apr. 4, 2013

(54) METHOD TO DETECT TISSUE DEGRADATION LEADING TO INFLAMMATION

(71) Applicants: Anna Blom, Malmo (SE); Kaisa Happonen, Malmo (SE); Dick Heinegård, Lund (SE); Tore Saxne, Lund (SE)

(72) Inventors: Anna Blom, Malmo (SE); Kaisa Happonen, Malmo (SE); Dick Heinegård, Lund (SE); Tore Saxne, Lund (SE)

(21) Appl. No.: 13/636,145

(22) PCT Filed: Mar. 30, 2011
(86) PCT No.: PCT/SE2011/050369

§ 371 Date: Dec. 3, 2012

(30) Foreign Application Priority Data
Mar. 31, 2010 (SE) 1050310-0

(51) Int. Cl. G01N 33/68

(52) U.S. Cl. CPC G01N 33/6893
USPC 435/7.92

(57) ABSTRACT

This invention relates generally to a method, an assay and a kit for determining a tissue degradation process that leads to inflammatory responses opening up for a vicious circle of increased tissue destruction. More specifically the invention relates to kits and methods for an assay that can analyzee human samples, for the presence of a COMP fragment complex that have activated complement exemplified by the complex between COMP and complement factor C3b or natural breakdown fragments of C3b.

### CROSS-REFERENCE TO RELATED APPLICATIONS

This application claims priority from PCT application PCT/SE2011/050369. filed Mar. 30, 2011, which claims priority from Swedish application 1050310-0 filed Mar. 31, 2010.

### FIELD OF THE INVENTION

The present invention relates generally to methods and products for determining a tissue degradation process and particularly events related to inflammation and propagation in joint disease. This invention relates to an assay that can be used to analyze serum, and other human samples (including but not limited to synovial fluid), for the presence of a COMP fragment complex that has activated the complement system exemplified by the complex between COMP and complement (actor C3b or natural breakdown fragments of C3b.

### BACKGROUND OF THE INVENTION

While there are more than 100 different types of arthritis, the most common are rheumatoid arthritis (RA) and osteoarthritis (OA) as well as spondylarthritides and juvenile idiopathic arthritis. About 1.3 million people in the U.S. have RA, nearly 10 times as many have OA.

Pathological conditions resulting in tissue degradation destroying joint structures such as cartilage constitute a major medical, social and economical problem. Of persons older than 65 years of age, about 500 out of 1,000 have arthritis. Tissue degradation processes are a result of the breakdown of tissue molecular constituents. This can be triggered by e.g. mechanical stress, toxic compounds or by inflammation leading to production of degradative enzymes. For this reason, determination of tissue degradation processes for the purpose of diagnosis, disease monitoring, treatment etc. can be performed by numerous methods. One way to determine degradation processes in connective tissue diseases, such as arthritic conditions, artheriosclerosis, degenerative joint conditions etc, is the detection of the presence of degradation products of the connective tissue components. This allows direct detection of the degradation process, compared to indirect methods as e.g. measuring increased amounts of leukocytes, which have been employed in the diagnosis of inflammatory processes such as arthritic conditions. Another parameter is the tissue loss observed late in the process that is detected by imaging methods such as X-ray or MRI.

Traditionally, the clinical diagnosis of arthritis is based on the patient's history, physical examination, in the case of RA laboratory tests particularly for certain antibodies and radiographs. The prognosis, treatment and clinical outcome of patients with arthritis are assessed by serial determinations. However, in order to minimize permanent tissue damage caused by pathological conditions involving cartilage degeneration, it is important to be able to diagnose such conditions at an early stage and particularly to assess risks for events that can propagate the process and enhance symptoms. Accordingly, during the last decade efforts have been made to find suitable biological markers that enable early detection of pathological cartilage degeneration.

One such biological marker is Cartilage Oligomeric Matrix Protein (COMP or Thrombospondin V). Elevated serum level of COMP has previously been associated with joint destruction in RA.

COMP is a structural component of cartilage which in the growing tissue appears to serve as a catalyst of collagen fibrillogenesis and in the older individual appears to have a structural role in maintaining tissue integrity. Further COMP is a pentameric glycoprotein with a predominant expression in cartilage. This protein is one of the main molecular markers for joint destruction, and elevated levels of COMP can be found both in the synovial fluid and in the serum of patients with active joint disease [1, 2]. COMP is found most abundantly in cartilage [3] and pressure loaded parts of tendon. In addition, some expression has been reported in synovial and dermal fibroblasts as well as in the blood vessel wall in atherosclerosis. Interestingly, elevated levels of COMP can he found in the blood of patients suffering from systemic sclerosis with skin involvement [4].

One of the main functions of COMP is to catalyze collagen fibrillogenesis and stabilize tissue structure by a direct interaction with collagen types I/II and to stabilize the collagen network via interactions with collagen IX and matrilins at the periphery of the collagen fibers. COMP has also been proposed to mediate attachment of chondrocytes to the extracellular matrix through interactions with cell-surface integrals. Structurally COMP is a pentamer consisting of five identical summits that axe linked together by a coiled coil structure close to the N-terminus. The N-terminus is followed by tour epidermal growth factor (EGF) domains, eight thrombospondin type 3 (TSP3) repeats and a globular C-terminus [5]. Mutations in COMP have been shown to lead to pseudoachondroplasia and multiple epiphyseal dysplasia.

The complement system is an array of factors that can rapidly be activated in defense as a part of the innate immune system. Complement activates inflammatory responses and recruits immune cells to the site. If also assists the adaptive immune system in e.g. clearing of damaged and dying cells, misfolded proteins, pathogens and foreign matters from an organism. Yet another important event is opsonisation of the target recognized by complement with C3b and its fragment which is a strong signal for phagocytosis and activation of B cells.

The complement system consists of a number of proteins normally circulating as inactive precursors (pro-proteins). Over 30 proteins and protein fragments make up the complement system, including glycosylated serum proteins and cell membrane receptors. These proteins are synthesized mainly in the liver, and they account for about 5% of the globulin fraction, of blood serum. When stimulated by one of several triggers, proteases in the system cleave specific proteins to release chemoattractant anaphylatoxins and initiate an amplifying cascade of further cleavages. The end-result of this activation cascade is massive amplification of the response with formation of factors active in cell recruitment and with final activation of the pore-forming membrane attack complex.

The complement system is a sensor of danger aiding in the removal of apoptotic and necrotic cells and immune-complexes as well as a defense mechanism against foreign pathogens. Uncontrolled complement activation can on the oilier hand contribute to several autoimmune disorders and pathological inflammatory conditions. Activation of complement has been shown to occur in the joints of patients suffering from RA and the involvement of complement in pathology of RA has been corroborated in animals models [6].

Three biochemical pathways activate the complement system; the classical complement pathway, the alternative complement pathway, and the mannose-binding lectin pathway, which merge at the level of C3-convertases that activate the main complement component C3.The classical pathway is typically triggered by immune complexes, whereas the lectin pathway is initiated by specific carbohydrate structures present on pathogenic surfaces. The alternative pathway is an autoactivation pathway that also serves as an amplification loop for the other two pathways. Recently, it has been confirmed that properdin can, directly activate the alternative pathway [7]. In all three pathways of complement activation the crucial step is the proteolytic conversion of the component C3 to C3b. Cleavage of C3b by enzymes of the complement cascades will allow the covalent attachment of C3b onto antigen surfaces via the thioester bond capturing either amino or hydroxyl group. This is the initial step in complement opsonisation, and subsequent proteolysis of the bound C3b by complement inhibitor factor I produces iC3b, C3c and C3dg, fragments that are recognized by different receptors, C3 is a complex and flexible protein consisting of 13 distinct domains. Comparison of C3b and C3c structures to C3 demonstrate that the molecule undergoes major conformational rearrangements with each proteolytic step, which exposes additional new surfaces of the molecule that can interact with cellular receptors and other ligands. Most inhibitors of the complement activation act at the level of C3b, the central component of the complement convertases.

Complement can also be triggered by a number of endogenous ligands. such as members of the small leucine-rich repeat protein (SLRP) family [8]. The SLRPs have several roles in cartilage, e.g. in contributing to the structural stability of the tissue. During pathogenic cartilage destruction. SLRPs are fragmented and released into the synovial fluid where they can interact with complement. This has been proposed to contribute to the local inflammatory milieu in joints of patients suffering from joint diseases such as RA and OA, where inflammation is prevalent.

As discussed above there is a need for better and improved methods and products for determining a tissue degradation process and the development of OA and RA, The present invention uses a novel approach to identify the specific process of complement activation/inflammation driven by products released as a result of the tissue destroying process. Complexes formed between molecules/fragments released from the tissue and complement factors resulting from complement activation are assayed by immunochemical methods in the invention herein that describes the assay of complexes between COMP and C3b or natural/further breakdown products of C3b.

In WO0138876 there is an assay described for a method of analyzing the presence of human COMP in a clinical sample using a sandwich-ELISA technology with two monoclonal antibodies produced from cell lines. The methods described in WO0138876 can partly be used in the present invention, which is hereby fully incorporated by reference, however there is no information therein on the concept now presented regarding a role of COMP in complement activation or complex formation with C3b.

In WO05116658 there is an assay described for determining a tissue degradation process by detection of COMP neoepitopes from the same group as the present invention. Parts of the methods described in WO05116658 can be used in the present invention, which is hereby fully incorporated by reference, however there is no information therein regarding also assaying complexes between COMP and another factor such as C3b.

US2008233113 relates to a method of inhibiting complement activation by C3b inhibitors in a subject but it does not relate to an assay for an agent activating complement and the resulting complexes between COMP and C3b. Neither is the procedure applied to showing that there is complement activation in the synovial fluid of both OA and RA relating to the COMP release.

WO2004031240 describes a method to inhibit inflammatory reactions m vivo, mote specifically the activation of the complement system. The invention consists of the identification and inhibition of a novel functional domain on the native third component of complement, C3, winch domain is essential for the activation of C3, but it does not relate to any agent accomplishing this activation nor an assay of complexes between COMP and C3b.

In WO2008154251 the invention concerns specific antibodies to C3b and the prevention and treatment of complement-associated disorders using such antibodies. However, the invention does not relate to an assay of complexes between COMP and C3b.

Excessive cartilage degradation during joint disease leads to molecular changes within the synovium. COMP-release from cartilage has been shown to be an early event in both RA and OA preceding radiologically observed cartilage damage. Elevated levels of COMP can therefore be detected both in the synovial fluid and in the serum of patients with active disease. Complement activation has been suggested to be one of the factors sustaining the inflammatory state in the joint especially since the discovery of the activation of the classical pathway of complement by certain cartilage proteoglycans. Complement activation products of both the classical and alternative pathways can be found in synovial fluids of patients with active RA, a scenario supported by several animal models showing the protective effect of deficiencies of complement proteins in arthritis disease models as well as therapeutic effects of complement inhibition [6].

Our invention is based on the fact that COMP-C3b complexes are present in both serum and synovial fluid of RA patients as an indication of COMP-induced complement activation in vivo. There is no correlation between the amount of COMP and COMP-C3b complexes in serum or synovial fluid, showing that only certain released fragments of COMP have complement activating properties or that there are other limiting factors. The COMP-levels in synovial fluid of RA patients are significantly higher than in serum whereas the COMP-C3b levels are somewhat lower compared to serum. One theory is that the events in blood reflect many joints which may contribute differently and another is that complement as well as total protein levels are in general much lower in synovial fluid that in serum and the availability of properdin and C3 might be a restricting factor.

The invention herein is based on the findings of the role of COMP in complement regulation and that COMP is able to induce complement activation through the alternative pathway.

By detecting COMP-C3b complexes in serum and not only COMP it is possible to identity active disease, for example RA or OA, in patients where circulating levels of COMP may remain in the normal range. These complexes are included in the method, in the assay and the kit according to the present invention.

The invention will also prove useful in identifying the role of complement activation driven by fragments released from the cartilage in other inflammatory conditions with COMP release, e.g. spondylarthritides and juvenile idiopathic arthritis, as well as other conditions like systemic sclerosis and tendon disease. This will provide an additional valuable tool for quickly establishing a diagnosis, selecting, and starting an early treatment of patients to avoid extensive joint destruction. The finding and tire invention e.g. a bioassay detecting covalent complexes between COMP and complement component C3b, or natural breakdown products of C3b, will detect a new biomarker for RA disease specifically useful for monitoring disease treatment. The assay will also likely identify a subgroup of RA patients particularly amenable to treatment with emerging complement inhibitors.

Thus it is an object of the present invention to identifying patients with active disease as well as identifying events causing local inflammation in the joint space of patients with active disease.

None of the above mentioned inventions and prior art, taken either singularly or in combination, describes (his invention or provides information that leads to this invention.

### SUMMARY OF THE INVENTION

Inflammation is a feature in most diseases. Previous findings indicate that molecules released from the tissue can affect complement activation as an important part in inflammation. Such molecules can be identified in body fluids. The current invention shows a new method to detect ongoing complement activation induced by the tissue destroying process. By detecting COMP-C3b complexes in serum, and not only COMP, it is possible to obtain a several fold more sensitive and specific detection of RA. By identifying such complexes in the synovial fluid of patients with joint disease it is possible to identify a local activation of inflammation resulting from the cartilage breakdown.

Blood and synovial fluid samples from patients with joint diseases, as for example RA, OA as well as tendinitis, systemic sclerosis and potentially certain stages of cardiovascular disease (including atherosclerosis) affecting major blood vessels, can be analyzed for contents of COMP-C3b complexes.

A primary object, of the present invention is to provide an assay that can be used to measure and to monitor the success of ongoing treatment regimes, i.e. the signal in the COMP-C3b assay follows disease activity in the patient.

Another object of the present invention is to provide an improved assay for identifying patients with RA.

An embodiment of the invention is an assay, as in the method of tire invention, where the clinical sample is used for measuring the complement activation in diseases with an inflammatory component and affecting connective tissues.

An embodiment of the invention is an assay, as in the method of tire invention, where the sample is used tot measuring the complement activation in suspected RA. and OA

An embodiment of the invention is an assay, as in the method of the invention, where the sample is used for measuring the complement activation in psoriatic arthritis, chronic juvenile arthritis and pelvospondylitis.

An embodiment of the invention is an assay, as in the method of tire invention, where the sample is used for measuring the complement activation in diseases with m inflammatory components and affecting connective tissues such as RA and OA, systemic sclerosis, tendinitis and cardiovascular disease including atherosclerosis.

An embodiment of the invention is an assay, as in the method of the invention, for monitoring the disease progression in patients with diseases with inflammatory components and affecting connective tissues such as RA and OA. systemic sclerosis, tendinitis and cardiovascular disease including atherosclerosis.

### BRIEF DESCRIPTION OF THE DRAWING

FIG. 1 shows (he levels of serum COMP-C3b complexes in various disease groups.

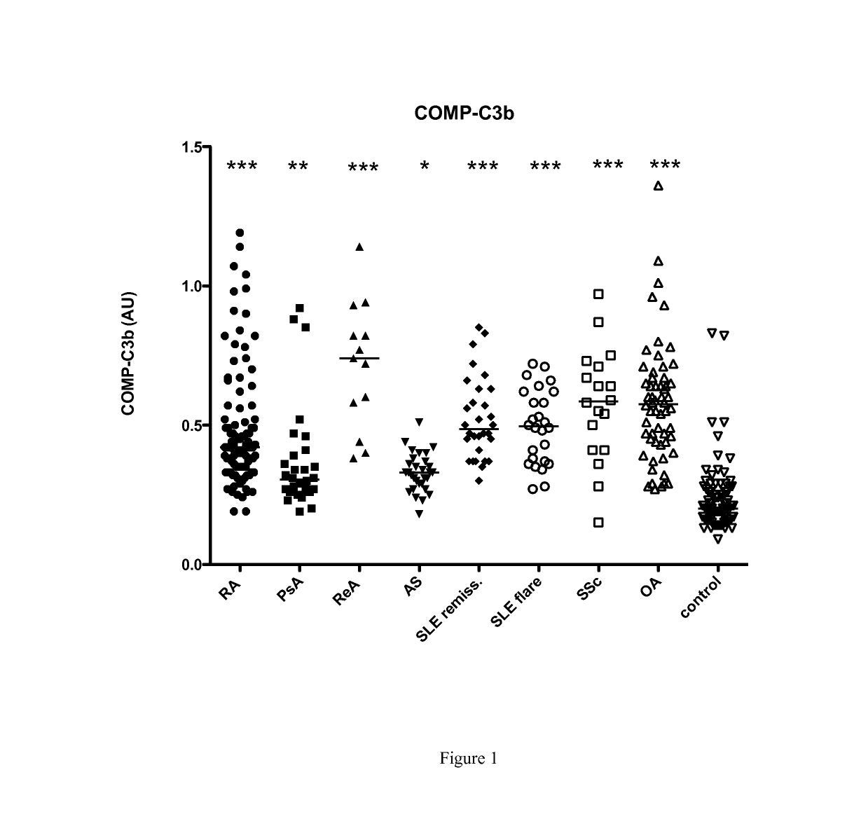

### DETAILED DESCRIPTION OF THE INVENTION

Inflammation is a component in most diseases. Previous findings indicate that molecules released from the tissue can affect complement activation as an important part in inflammation We have found that COMP is able to activate the alternative pathway of the complement system mainly through an interaction with properdin, which might direct complement attack to surfaces with exposed COMP. A pentameric structure of COMP (pCOMP) was not necessary for promoting the binding of properdin, supporting the in vivo finding that released fragment's of cleaved COMP in serum have complement activating properties. Interestingly, C3 and C3b were also found to internet directly with COMP. This raises the question of whether the effect of COMP is to stabilize the alternative C3-convertase through interacting with both C3b and properdin or more simply to target complement activation by recruiting properdin and therefore providing a platform for convertase assembly

The invention is aimed to be used to detect inflammation in general in a patient.

The invention relates for example to a kit for determining a tissue degradation process by a method according to the invention, comprising the following components:
i) A first component comprising one or more antibodies or another ligand catching one of the components forming the COMP-C3b complex, exemplified in the present application by antibodies to COMP according to the procedure described in WO0138876/AU9904236, which is incorporated herein. The antibodies can be ligands for COMP, antibody fragments, such as Fab or F(ab)′2 including such identified by the phage-display methods or intact antibodies binding to COMP epitopes,
ii) A second component comprising the detector in the form of one or more antibodies binding to the other component of the complex, exemplified it) the present application by antibodies to complement factor C3b or natural breakdown products or fragments thereof,
iii) A third component comprising means for detecting whether one or more antibodies have reacted with one or more epitopes of the fragments as described above. This cart be achieved by using a labelled detector antibody, or a labelled ligand binding to the detector antibody. In typical approaches the final detection is by commonly used approaches e.g. such including measuring activity of bound enzymes or any other probe.

Furthermore the invention can also relate to a kit for determining a tissue degradation process by a method according to the invention, comprising tire following components:

i) A first component providing an antibody, for example a monoclonal antibody or another ligand, directed against human COMP, either in solution or bound to a solid phase carrier, such as a well in a plate,

ii) adding a clinical sample suspected to contain human COMP-C3b fragment complex to a first antibody of step i) and incubating the resulting mixture in aqueous solution;

iii) adding a second antibody, for example a monoclonal antibody or another ligand, directed against human C3b or fragments thereof to the mixture of step ii), said second antibody comprising a label emitting a detectable and quantifiable signal and quantifying the signal from the label, said signal being a measurement of the concentration of human COMP-C3b complex in said sample.

The antibodies of the invention are optionally labeled with e.g. an enzyme, a radioactive, fluorescent or luminescent label in order to measure the level of bound antibody to the COMP-C3b complex present in the sample.

In the sandwich assay, the component bound to the solid phase can either be the first or the second component. In the example given above, the first component is bound to the solid phase. In another example of the sandwich assay, the second component comprising the antibodies of the present invention is bound to a solid phase, such as e.g. a well in a plate. The presence of COMP-C3b fragments in a sample can then be detected by first adding the sample to the well in the plate. If one or more complexes containing COMP-C3b fragments are present in the sample, the fragments will be bound to the solid phase via the antibodies, which bird to the COMP-C3b complex. The presence of fragments containing C3b epitopes can. then be measured by adding the first component comprising one or more substances, which bind to one or more fragments. The one or more substances are optionally labelled with e.g. an enzyme or a radioactive or fluorescent label in order to measure the level of bound substance to the fragments present in the sample. Also a labeled ligand, such as an antibody, reacting with the second antibody can be used tor detection.

The invention will provide information on the synovial fluid level of complement activation induced by the process of cartilage degradation in joint disease. Serum analyses will provide information on the level of complement activation induced by the released fragments of COMP and better identity patients suffering from RA and OA, In particular information on the role of matrix components in eliciting inflammation will provide an important component in evaluating future therapeutic endeavors as well as in selecting patients for such therapy.

The invention will provide novel means to define RA patients with active disease and will include patients before current diagnosis can be made in the preclinical phase.

The invention will define disease activity even, when tissue destruction is of too low an intensity to be detected by conventional current assays, including that for COMP.

The invention will define high risk patients with a more intense disease progression.

The invention will define patients having a higher risk of develop degenerative joint disease after trauma by analyses of synovial fluid.

The invention will define complement activation in psoriatic arthritis, chrome juvenile arthritis and pelvospondylitis.

The invention will identify processes in the spine leading to back pain by analyses of serum samples. The rationale is that COMP fragments released will activate complement to induce the inflammatory process in turn yielding pain. The complexes demonstrating complement activation will provide direct measures of the process.

### Example 1

Blood samples are collected by venipuncture and are allowed to clot. Serum is separated by centrifugation. The samples are then diluted 1:10 in sample diluent (0.05 M Tris-HCl, pH 7.5, 0.90 percent (wt) NaCl, 1 percent bovine serum albumin, 0.05 percent Tween 20, 0.15 percent Kathone CG, 0.01 percent tartrazine, 0.001 M CaCl₂, 0.01 percent bovine IgG, filtered using a 0.45 micro m filter) (12 μL (microliter) sample to 108 μL (microliter) sample diluent).

Synovial fluid is collected by joint aspiration and immediately centrifuged to remove cells and any particles. The synovial fluid is diluted 1:10 in sample diluent as the serum samples.

Each determination is performed in duplicate for references and unknown samples. A polystyrene 96-well microliter plate, wherein the monoclonal antibody produced by cell line DSM ACC2406 is immobilised in the wells, is used. 50 μL of unknown sample or reference sample (in our case we used the 1.7 U/l calibration control as a reference) is added to the wells and the plate is incubated, for 120 min on a rotating plate at room temperature. After washing 4 times with 350 μL washing buffer (0.14 M NaCl, 0.003 M KCl, 0.05% Tween 20, 0.01 M phosphate buffer pH 7.4), 50 μL conjugate solution diluent (0.05 M Tris-base pH 7.5, 0.9 percent (wt) NaCl, 0.001 M CaCl2×2H2O, 1 percent BSA, 0.05 percent Tween 20, 0.15 percent. Kathon CG, 0.03 percent patent blue, 0.01 percent bovine IgG, 0.005 percent heterophilic blocking reagent-1) containing the C3b recognizing antibody C7761 from Sigma (St. Louis, Mo. 63178, UNITED STATES ) (diluted 1:3000) is added to all wells and the plate is incubated on a rotator for 60 minutes at room temperature. The plate is washed 4 times with washing buffer and 50 μL of rabbit anti-goat HRP (P0449 from Dako Cytomation, diluted 1:2000) in conjugate solution diluent is added to each well. The plate is incubated 60 min at RT (room temperature) on a rotator after which if is washed 4 times with washing buffer as above. 200 μL of 3,3′, 5,5′-tetramethylbenzidine (1 mM) is added to each well and the plate is incubated for 3 min and 30 s at room temperature. The color reaction is stopped by adding 50 μL 0.5 M H₂SO₄ to each well. The absorbance at 450 nm is measured. The absorbance of the reference sample is set to 1 and the readouts for the patient samples are normalized against the reference.

### Example 2

As in Example 1 but also a positive pool of sera is used, representing one or more levels of complex as a direct standard to obtain quantitative measures of the COMP-C3b levels in the unknown samples.

### REFERENCES

1 Morozzi, G., Fabbroni, M., Bellisai, F., Pucci, G. and Gafeazzi, M., Cartilage oligomeric matrix protein level in rheumatic diseases; potential use as a marker for measuring articular cartilage damage and/or the therapeutic efficacy of treatments. Ann N Y Acad Sci 2007. 1108: 398-407.
2 Saxne, T. and Heinegärd, D., Cartilage oligometric matrix protein: a novel marker of cartilage turnover detectable in synovial fluid and blood. Br J Rheumatol 1992. 31: 583-591.
3 Hedbom, E., Antonsson, P., Hjerpe, A., Aeschlimann, D., Paulsson, M., Rosa-Pimentel, E., Sommarin, Y., Wendel, M., Oldberg, A. and Heinegärd, D., Cartilage matrix proteins. An acidic oligomeric protein (COMP) detected only hi cartilage. J Biol Chem 1992. 267; 6132-6136.
4 Hesselstrand, R., Kassner, A., Heinegärd, D. and Saxne, T., COMP: a candidate molecule in the pathogenesis of systemic sclerosis with a potential as a disease marker. Ann Rheum Dis 2008. 67: 1242-1248.
5 Oldberg, A., Antonsson, P., Lindblom, K. and Heinegärd, D., COMP (cartilage oligomeric matrix protein) is structurally related, to the thrombospondins. J Biol Chem 1992. 267:22346-22350.
6 Okroj, M., Heinegärd, D., Holmdahl, R. and Blom, A. M., Rheumatoid arthritis and the complement system. Ann Med 2007. 39; 517-530.
7 Spitzer, D., Mitchell, L. M., Atkinson, J. F and Hourcade, D. E., Properdin can initiate complement, activation by binding specific target surfaces and providing a platform forde novo convertase assembly. J Immunol 2007. 179: 2600-2608.
8 Sjöberg, A. P., Trouw, L. A. and Blum, A. M., Complement activation arid inhibition: a delicate balance. Trends Immunol 2009. 30: 83-90.

1. A method of determining a tissue degradation process that leads to complement activation comprising detecting the presence of a complex between human COMP and one or more complement factors, or fragments thereof.

2. The method according to claim 1 wherein detecting the presence of a complex comprises comprising the following steps:
a) providing a sample from a patient;
b) analyzing the sample;
c) detecting the presence of a complex between human COMP and complement factors in the sample.

3. The method according to any of the preceding claims wherein the sample is a clinical sample, preferably from serum or synovial fluid.

4. The method according to claim 1 where the complement factor is human C3b, or fragments thereof.

5. An assay wherein a sample as defined in claim 1 wherein the complement factor is human C3b or fragments thereof is used for measuring or for monitoring the complement activation in diseases with an inflammatory component and affecting connective tissues.

6. The assay as in claim 5 where the sample is used for measuring the complement activation in suspected RA and OA

7. the assay as in claim 5 where the sample is used for measuring the complement activation in psoriatic arthritis, chronic juvenile arthritis and pelvospondylitis.

8. the assay according to claim 5, wherein the sample is used for measuring the complement activation in diseases with an inflammatory components and affecting connective tissues such as RA and OA, systemic sclerosis, tendinitis and cardiovascular disease including atherosclerosis.

9. An the assay according to claim 5, wherein the sample is used for monitoring the disease progression in patients with diseases with inflammatory components and affecting connective tissues such as RA and OA, systemic sclerosis, tendinitis and cardiovascular disease including atherosclerosis.

10. A kit for determining a tissue degradation process that leads to complement activation comprising detecting in a sample the presence of a complex between human COMP and complement factors.

11. A The kit according to claim 10 wherein the kit comprises:
a) one or more antibodies, or another ligand, binding to one of the components forming the COMP-C3b complex;
b) a detector in the form of one or more antibodies binding to the other component of the complex; and
c) means for detecting whether one or more antibodies have reacted with one or more epitopes of the fragments as described above.

12. (canceled)

13. A The kit according to claim 10 where the complement factor is human C3b or fragments thereof.

14. A The kit according to claim 10 wherein the kit is to be used for a sample suspected of containing human COMP-C3b fragment complex and the kit comprises:
a) an antibody, preferably a monoclonal antibody or another ligand, directed against human COMP, being in solution or bound to a solid phase carrier;
b) a second antibody, preferably a monoclonal antibody or another ligand, directed against human C3b or fragments thereof; said second antibody comprising a label emitting a detectable and quantifiable signal; and
c) means to quantify the signal from the label, said signal being a measurement of the concentration of human COMP-C3b complex in said sample.

15. The method according to claim 2 where the complement factor is human C3b, or fragments thereof.

16. The assay of claim 5, wherein detecting the presence of a complex comprises the following steps: a) providing a sample from a patient; b) analyzing the sample; and c) detecting the presence of a complex between human COMP and complement factors in the sample.

17. The assay of claim 5, where the complement factor is human C3b, or fragments thereof.

18. The kit according to claim 11 where the complement factor is human C3b or fragments thereof.

19. The kit according to claim 11 wherein the kit is to be used for a sample suspected of containing human COMP-C3b fragment complex and the kit comprises:
a) an antibody, preferably a monoclonal antibody or another ligand, directed against human COMP, being in solution or bound to a solid phase carrier;
b) a second antibody, preferably a monoclonal antibody or another ligand, directed against human C3b or fragments thereof; said second antibody comprising a label emitting a detectable and quantifiable signal; and
c) means to quantify the signal from the label, said signal being a measurement of the concentration of human COMP-C3b complex in said sample.
